(19) United States
(12) Patent Application Publication
(10) Pub. No.: US 2011/0141016 A1
(43) Pub. Date: Jun. 16, 2011

(54) MOUSE FOR MEASURING CONSISTENCY OF BLOOD OXYGEN

(76) Inventors: Chun Hao LU, Rende Hsiang (TW); Cheng Chi Tai, Rende Hsiang (TW); Jiun Hung Lin, Rende Hsiang (TW)

(21) Appl. No.: 12/638,316

(22) Filed: Dec. 15, 2009

(51) Int. Cl. G09G 5/08; A61B 5/05

(52) U.S. Cl. 345/163; 600/345

(57) ABSTRACT

A mouse for measuring consistency of blood oxygen includes a mouse case, on which two transmitters, a transmitting circuit, a receiver, a receiving feedback circuit, a filtrate amplifying circuit, and a calculating unit are disposed. The transmitting circuit serves to control the transmitters to alternately radiate a red glow and an infrared light to the receiver. The receiving feedback circuit applies to convert them into an electrical signal, and the filtrate amplifying circuit obviates any promiscuous frequency existing in the electrical signal and amplifies the electrical signal. The calculating unit accordingly computes the signal to obtain the value of consistency of blood oxygen, which would thence be outputted from a communication interface for informing users about the consistency of blood oxygen, so as to prevent the discomfort suffered by the occurrence of oxygen deficiency in blood.

### BACKGROUND OF THE INVENTION

1. Field of the Invention

The present invention relates to a mouse for measuring consistency of blood oxygen, in particular to one which can freely monitor the consistency of blood oxygen for preventing the discomfort incurred by the oxygen deficiency in blood.

2. Description of the Related Art

A typical mouse normally includes multiple buttons disposed on the mouse case and electrically communicated with a controlling circuit inside the case; by such a combination, the user could operate the buttons for transmitting signals indicative of moving, selecting or confirming actions to the controlling circuit, from which the signals would be further send to a CPU through a wired or wireless communication interface. A normal use of computer is attained.

In virtue of the typical mouse lack of variations, an improved mouse with enhanced functions is disclosed by R.O.C. Patent No. M342549, which essentially includes at least one heat sensor, a micro-processing module coupled to the mouse, a laser instructing module coupled to the micro-processing module, a touch-control module coupled to the micro-processing module for users to manipulate the laser instructing module or the CPU, and a programming module for proceeding a list of programmed instructions. Wherein, the heat sensor detects thermal from the body heat and sends a detected signal to the micro-processing module for rousing it out of a sleeping state. The touch-control module generates a touching signal and transmits it to the micro-processing module, so that the micro-processing module could alternatively control the laser instructing module or generate a controlling signal to the CPU in light of the touching signal. The programming module serves to define multiple functional labels for user's operation and thence creates the touching signal, so as to increases the functions and utilizations of the mouse. However, such configuration raises some disadvantages as follows:

In view of the computer are usually placed in a closed environment, the computer users worked with great dedication would ignore the gravity of lack of well ventilation and facilely lead to the reduction of the blood oxygen in the body, thence the users would become tired or discomfort.

### SUMMARY OF THE INVENTION

The primary object of the present invention is to solve the conventional problems of the typical mouse and to provide a mouse for measuring consistency of blood oxygen.

The mouse in accordance with the present invention includes a mouse case, in which a controlling circuit is disposed; wherein, the mouse case includes a communication interface and at least one controlling button; the communication interface and the controlling button are electrically connected to the controlling circuit. Further, two transmitters are disposed on the mouse case for respectively emitting a red glow with wavelength at 660 nm and a ray of infrared light with wavelength at 940 nm. A transmitting circuit is disposed inside the mouse case and electrically connected to the transmitters for controlling the transmitters to alternately emit the red glow and the infrared light. A receiver is disposed on the mouse case and located on a route where the red glow and the infrared light is thrown back, so as to receive the reflected red glow and the infrared light. A receiving feedback circuit is disposed inside the mouse case and electrically connected to the receiver and the transmitting circuit, which serves to convert light signals from the reflected red glow and the infrared light into an electrical signal and thence send a feedback signal to the transmitting circuit. A filtrate amplifying circuit is disposed inside the mouse case and electrically connected to the receiving feedback circuit, thereby precluding the promiscuous frequency existing in the electrical signal and amplifying the electrical signal. A calculating unit is disposed inside the mouse case and electrically connected to the filtrate amplifying circuit and the communication interface respectively, thence receiving the electrical signal and computing the values of the consistency of blood oxygen and the heartbeat. These values would be sent out from the communication interface.

Preferably, the communication interface is either a wire cable or a wireless transceiver.

Preferably, the mouse case is provided with a screen which is electrically connected to the calculating unit, thereby outputting the value of the consistency of blood oxygen or the heartbeat.

Preferably, the transmitters and the receiver are concurrently disposed either at a top portion or a side portion of the mouse case.

It is well know that blood mainly contains deoxygenate hemoglobin (Hb), oxygenate hemoglobin (Hb02), carboxyhemoglobin, carbon dioxide hemoglobin, hemoglobin derivatives, leukocytes (white cells), and platelet. Wherein, Hb and Hb02 are critical components impinging on the consistency of blood oxygen and possess distinct properties regarding to the absorption spectroscopy within the red glow and the infrared light ranged from 600 nm to 1000 nm; namely the absorption coefficient of Hb is greater than that of Hb02 within the range of the red glow, and the reverse of which is performed within the range of infrared light, shown in the figure of light absorption spectroscopy between Hb and Hb02 as published in M. H. Smith, “Optimum wavelength selection for retinal vessel oximetry,” Appl. Opt. 38, 258-267 (1999). In the present invention, the red glow with wavelength at 660 nm and the infrared light with wavelength at 940 nm are preferably adopted as an optical signal for detecting the consistency of blood oxygen.

Accordingly, the present invention has advantages set forth below:
1. As mentioned supra, Hb02 and Hb in blood includes distinct absorption spectroscopy in the place of red glow and infrared light ranged between 600 nm and 1000 nm and of further properties that, the optical measurement is normal to provide the living organization with features of non-invasion, reliability, and successive monitoring. Therefore, present invention utilizes the red glow with wavelength at 660 nm and the infrared light with wavelength at 940 nm for an optical detecting concerns an easy working and higher reliability.
2. The present invention serves to compute the heartbeat for users to be freely aware of the condition of consistency of blood oxygen and the heartbeat, so as to prevent the discomfort suffered by the occurrence of oxygen deficiency in blood or abnormal heartbeat.

### BRIEF DESCRIPTION OF THE DRAWINGS

FIG. 1 is a perspective view showing the present invention;

FIG. 2 is a flow chart showing the present invention in operation;

FIG. 3 is a schematic view showing the present invention in a wired communication

FIG. 4 is a schematic view showing the present invention in a wireless communication; and

FIG. 5 is schematic view showing a further preferred embodiment of the present invention.

### DETAILED DESCRIPTION OF THE PREFERRED EMBODIMENTS

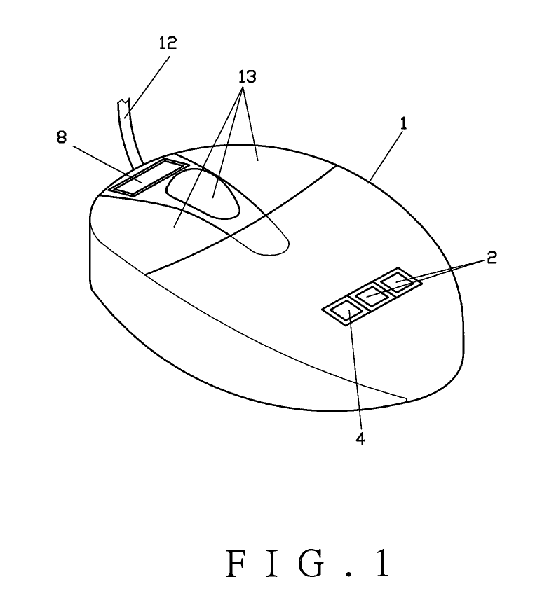

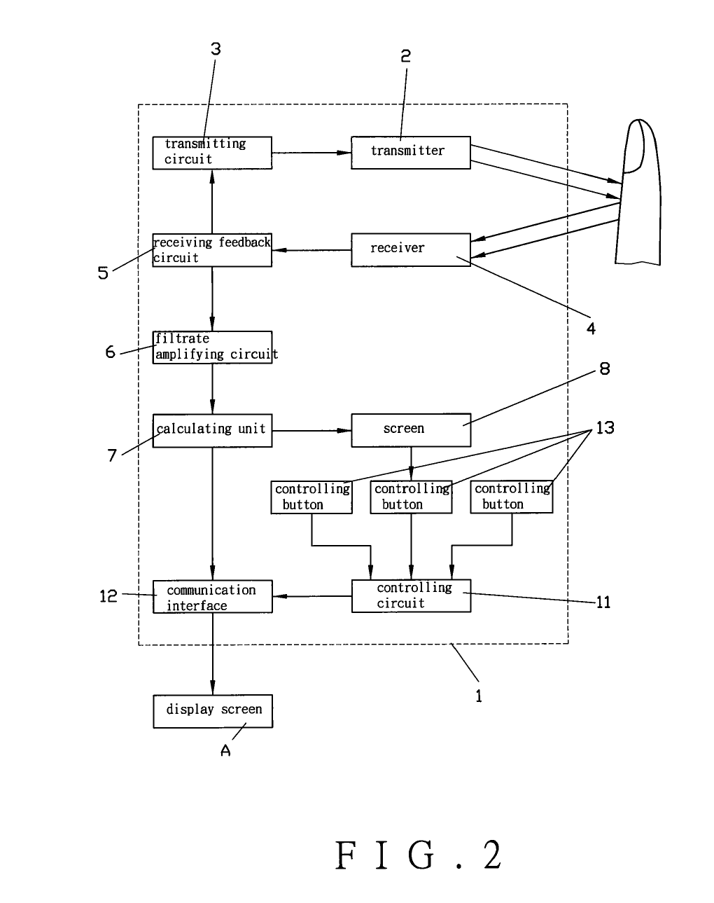

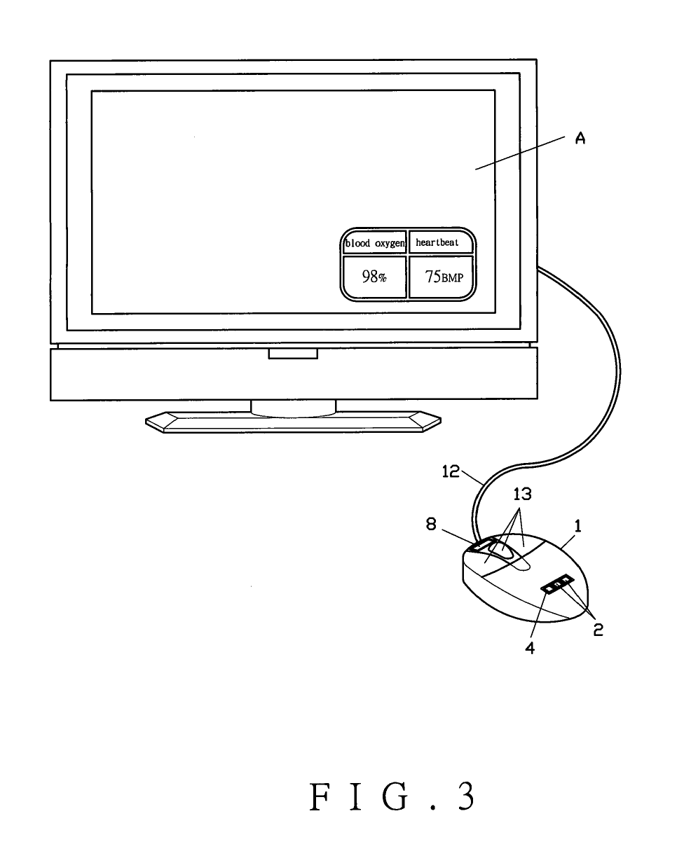

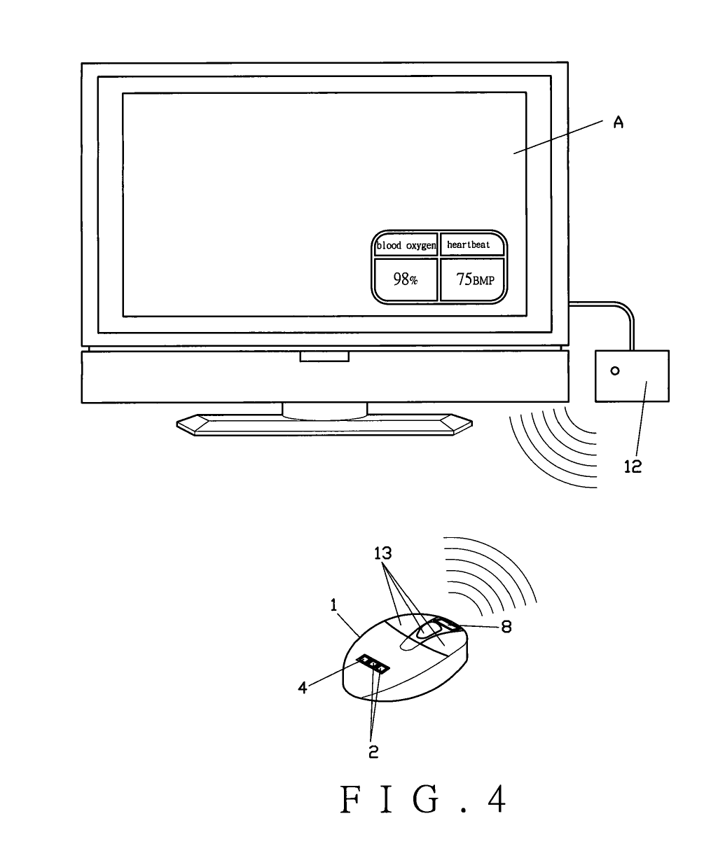

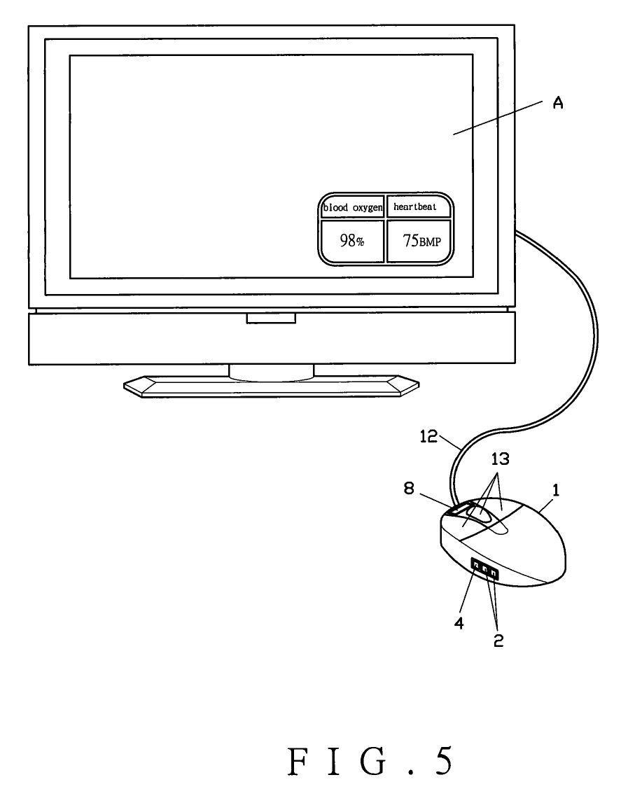

FIGS. 1 and 2 show a mouse of a preferred embodiment of the present invention for measuring the consistency of blood oxygen, which includes a mouse case 1, in which a controlling circuit 11 is disposed; wherein, the mouse case 1 includes a communication interface 12 and at least one controlling button 13; the communication interface 12 and the controlling button 13 are electrically connected to the controlling circuit 11. Further, the communication interface 12 could be either a wire cable as shown in FIG. 3 or a wireless transceiver as shown in FIG. 4. In addition, two transmitters 2 are disposed on the mouse case 1 for respectively emitting a red glow with wavelength at 660 nm and a ray of infrared light with wavelength at 940 nm. A transmitting circuit 3 is disposed inside the mouse case 1 and electrically connected to the transmitters 2 for controlling the transmitters 2 to alternately emit the red glow and the infrared light. A receiver 4 is disposed on the mouse case 1 and located on a route where the red glow and the infrared light is thrown back, so as to receive the reflected red glow and the infrared light. A receiving feedback circuit 5 is disposed inside the mouse case 1 and electrically connected to the receiver 4 and the transmitting circuit 3, which serves to convert light signals from the reflected red glow and the infrared light into an electrical signal and thence send a feedback signal to the transmitting circuit 3. A filtrate amplifying circuit 6 is disposed inside the mouse case 1 and electrically connected to the receiving feedback circuit 5, thereby precluding the promiscuous frequency existing in the electrical signal and amplifying the electrical signal. A calculating unit 7 is disposed inside the mouse case 1 and electrically connected to the filtrate amplifying circuit 6 and the communication interface 12 respectively, thence receiving the electrical signal and computing the values of the consistency of blood oxygen and the heartbeat. These values would be sent out from the communication interface 12. Moreover, the mouse case 1 includes a screen 8 electrically connected to the calculating unit 7, thereby directly outputting the values of the consistency of blood oxygen or the heartbeat. Additionally, the transmitters 2 and the receiver 4 are concurrently disposed either at a top portion of the mouse case 1 as shown in FIGS. 3 and 4 or at a side portion of the mouse case 1 as shown in FIG. 5.

Referring to FIGS. 2 to 5, in use, users could operate the controlling buttons 13 for transmitting signals indicative of moving, selecting or confirming instructions to the controlling circuit 11, from which the signals would be further outputted through the communication interface 12 for a normal use. When users grasp the mouse case 1, the transmitting circuit 3 would trigger the transmitters 2 to radiate a red glow with wavelength at 660 nm and an infrared light with wavelength at 940 nm in turn. The red glow and the infrared light would thence infiltrate into the human body. Such action leads to the body absorbing partial rays of the red glow and the infrared light, refracting partial rays of the same, and reflecting partial rays of the same to the receiver 4. The receiver 4 proceeds to receive light signals from the reflected red glow and the infrared light and sends them to the receiving feedback circuit 5 for converting the light signals into an electrical signal and thence transmitting to the filtrate amplifying circuit 6. A feedback signal back would be generated for returning back to the transmitting circuit 3. The transmitting circuit 3 controls the alternate emission of the red glow and the infrared light in light of the feedback signal. Furthermore, when the filtrate amplifying circuit 6 receives the electrical signal, it follows to preclude traces of promiscuous frequency existing in the light signals involving the reflected red glow and infrared light from any non-Hb02 and non-Hb and further to expand the electrical signal involving the reflected red glow and infrared light from Hb02 and Hb. Thereafter, the electrical signal is sent to the calculating unit 7 for computing the values of the consistency of blood oxygen or the heartbeat. These values would be outputted by the screen 8 or be sent out from the communication interface 12 so as to be exposed to a display screen A, so that users can be freely notified with respect to the consistency of blood oxygen and the heartbeat, so as to prevent the discomfort suffered by the occurrence of abnormal heartbeat or oxygen deficiency in blood, i.e. anoxia.

What is claimed is:

1. A mouse for measuring consistency of blood oxygen, comprising:
a mouse case having a controlling circuit therein, said mouse case including a communication interface and at least one controlling button, said communication interface and said controlling button being electrically connected to said controlling circuit; characterized in that said mouse further including:
two transmitters disposed on said mouse case;
a transmitting circuit disposed inside said mouse case and electrically connected to said transmitters;
a receiver disposed on said mouse case;
a receiving feedback circuit disposed inside said mouse case and electrically connected to said receiver and said transmitting circuit;
a filtrate amplifying circuit disposed inside said mouse case and electrically connected to said receiving feedback circuit; and
a calculating unit disposed inside said mouse case and electrically connected to said filtrate amplifying circuit and said communication interface, respectively.

2. The mouse as claimed in claim 1, wherein said communication interface is either a wire cable or a wireless transceiver.

3. The mouse as claimed in claim 1, wherein said mouse case is provided with a screen which is electrically connected to said calculating unit.

4. The mouse as claimed in claim 1, wherein said transmitters and said receiver are disposed either at a top portion or a side portion of said mouse case.
